(19) United States
(12) Patent Application Publication
(10) Pub. No.: US 2017/0152536 A1
(43) Pub. Date: Jun. 1, 2017

(54) METHOD FOR TRANSFORMING LEVOGLUCOSENONE INTO 4-HYDROXYMETHYN BUTYROLACTONE OR 4-HYDROXYMETHYL BUTENOLIDE

(71) Applicants: INSTITUT NATIONAL DE LA RECHERCHE AGRONOMIQUE, Paris Cedex 07 (FR); INST SCIENCES IND VIVANT ENVIRONNEMENT, Paris 5 (FR)

(72) Inventors: Florent Allais, Reims (FR); Amandine Flourat, Reims (FR); Aurélien Peru, Reims (FR); Andreia Teixeira, Reims (FR); Fanny Brunissen, Reims (FR); Henry Eric Spinnler, Sevres (FR)

(21) Appl. No.: 15/307,712

(22) PCT Filed: Apr. 29, 2015
(86) PCT No.: PCT/EP2015/059323

§ 371 Date: Nov. 22, 2016

(30) Foreign Application Priority Data
Apr. 30, 2014 (FR) 1453957

(51) Int. Cl. C12P 17/04

(52) U.S. Cl. CPC C12P 17/04

(57) ABSTRACT

The disclosure relates to a method for transforming levoglucosenone into 4-hydroxymethyl butyrolactone or 4-hydroxymethyl butenolide, comprising a step involving the oxidation of the levoglucosenone, or dihydrolevoglucosenone obtained by hydrogenation of levoglucosenone, by bringing a solution of levoglucosenone or dihydrolevoglucosenone in a solvent into contact with a lipase in the presence of an oxidizing agent and an acyl donor compound. The oxidation step is followed by a step involving the hydrolysis of the reaction mixture obtained and, if necessary, a step involving the hydrogenation of the compound obtained at the end of the hydrolysis step.

### CROSS-REFERENCE TO RELATED APPLICATIONS

This application is a national phase entry under 35 U.S.C. §371 of International Patent Application PCT/EP2015/059323, filed Apr. 29, 2015, designating the United States of America and published as International Patent Publication WO 2015/165957 A1 on Nov. 5, 2015, which claims the benefit under Article 8 of the Patent Cooperation Treaty to French Patent Application Serial No. 1453957, filed April 30, 2014.

### TECHNICAL FIELD

The invention relates to a method for transforming levoglucosenone into 4-hydroxymethyl butyrolactone or 4-hydroxymethyl butenolide (Ia).

### BACKGROUND

To solve the problem of dependency on fossil fuels, methods of producing chemical compounds from biomass have been developed over the last decades. In particular, lignocellulose biomass has become one of the organic sources of carbon compounds. Levoglucosenone, with the following formula:

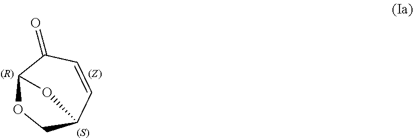

is one of the most interesting products that may be obtained from biomass, in particular, by the flash pyrolysis of cellulose.

Levoglucosenone is commonly used as a starting material for the synthesis of different compounds of interest, in particular, 4-hydroxymethyl butenolide, with formula (IIa) and 4-hydroxymethyl butyrolactone, with formula (IIb):

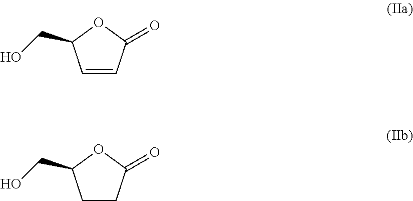 [SMILES: O=C1C=C[C@@H](CO)O1 ; O=C1CC[C@@H](CO)O1]

These compounds are of particular interest since they form (chiral) asymmetric intermediates with a high added value that are often used in the agro-food industry for the production of fragrances and flavors, or in the pharmaceuticals industry for the production of the active ingredients in drugs, taking advantage of their lactone ring or chiral center.

Levoglucosenone is transformed into 4-hydroxymethyl butenolide in a conventional manner in two successive steps. The first step consists of a Baeyer-Villiger oxidation reaction to form a formate intermediate, followed by an acid hydrolysis step to form 4-hydroxymethyl butenolide. To obtain 4-hydroxymethyl butyrolactone, the levoglucosenone is first subjected to a catalytic hydrogenation step to form dihydrolevoglucosenone, which then undergoes the steps in the transformation described above with reference to levoglucosenone.

Methods for the oxidation of levoglucosenone or dihydrolevoglucosenone have been described in the prior art.

By way of example, U.S. Pat. No. 4,994,585 describes the oxidation of levoglucosenone with a peracid, such as m-chloroperbenzoic acid or peracetic acid, in an organic solvent; and U.S. Pat. No. 5,112,994 describes a method for the synthesis of 4-hydroxymethyl butyrolactone from dihydrolevoglucosenone in which the oxidation is also carried out by a peracid in an organic solvent. However, to obtain a high yield, these reactions have to be carried out for a long period of time, from one or two days.

Paris et al., in Green Chemistry, 2013, 15:2101-2109, described the oxidation of levoglucosenone by metal catalysts such as the aluminium zeolites or tin, in the presence of an oxidizing agent. However, such methods may take a long time to implement, with certain catalysts proposed, to obtain a satisfactory rate of conversion of levoglucosenone. Aluminium zeolites provide high conversion rates within four hours. However, this time doesn't take into account the time required for the preparation of the catalyst. In addition, the latter is potentially toxic.

Moreover, with a goal to better protection of the environment through the use of less toxic substances, the prior art proposed the oxidation of cyclic ketones by Baeyer-Villiger reaction using, as a catalyst, a lipase immobilized on a carrier, in particular, grafted porous silica. In particular, such a method is described by Drozdz et al. in Applied Catalysis A: General, 2013, 467:163-170. As described in this document, this method is satisfactory, in terms of rate of conversion of cyclic ketone and reaction time, for ketones with a high ring strain such as cyclobutanone or cyclopentanone, but only for cyclic ketones of this type. On the contrary, with cyclic ketones with low ring strain, such as cyclobutanone or cyclopentanone, but only for cyclic ketones of this type. On the contrary, for cyclic ketones with a high ring strain such as cyclohexanone or cycloheptanone, such an oxidation reaction is not very efficient, since it provides a low rate of conversion and/or in a very long time. Such a difference in reactivity according to the ring strain, in particular, related to the number of members in the ring, is well known to the person skilled in the art, whose general knowledge in the field are, in particular, described in the document by Wiberg in Angew. Chem. Int. Ed. Engl., 1986, 25:312-322.

Levoglucosenone and dihydrolevoglucosenone both have a low ring strain, similar to that of cyclohexanone or cycloheptanone. As a result, such results divert the person skilled in the art from carrying out the oxidation of these compounds with acceptable times and yields, using the method described in the document. On the contrary, they encourage a search for alternative solutions.

### BRIEF SUMMARY

Now, in a totally unexpected way, it was noted that the oxidation of levoglucosenone or dihydrolevoglucosenone by enzyme catalysis, by means of a lipase in the presence of an oxidizing agent and an acyl donor agent, provides especially high rates of conversion in especially short reaction times, to form, after a subsequent step of hydrolysis, 4-hydroxymethyl butyrolactone or 4-hydroxymethyl butenolide.

The disclosure is thereby aimed at overcoming the disadvantages of the methods proposed by the prior art for the transformation of levoglucosenone into 4-hydroxymethyl butyrolactone or into 4-hydroxymethyl butenolide, in particular, those described above, by proposing a method that provides high rates of conversion of levoglucosenone, in less time, not exceeding several hours, and by using non-toxic environmentally friendly reagents.

An additional aim of the disclosure is that this method is economical to use.

Thereby, the disclosure relates to a method for transforming levoglucosenone (called LGO) into a compound with the following general formula (II):

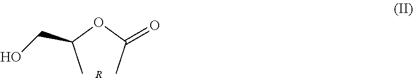

where R represents —CH═CH— or —CH₂—CH₂—.

More specifically, when R represents —CH═CH—, the compound in formula (II) is 4-hydroxymethyl butenolide (called HBO), with the following formula (IIa):

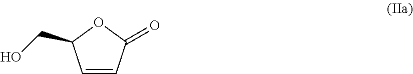

When R represents —CH₂—CH₂—, the compound in formula (II) is 4-hydroxymethyl butyrolactone (called HLO), with the following formula (IIb):

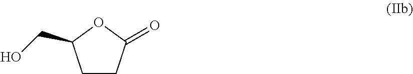

According to the disclosure, this method comprises the following successive steps:
a) If applicable, to obtain a compound with general formula (II) where R represents —CH₂—CH₂—, the first step involves the hydrogenation of the levoglucosenone (Ia) in order to form the dihydrolevoglucosenone of formula (Ib):

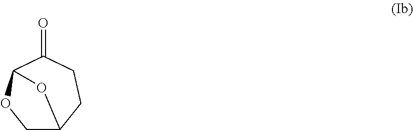

This step may be carried out by any conventional method in itself, in particular, by catalytic hydrogenation in the presence of palladium on charcoal.
b) Oxidation of the levoglucosenone or dihydrolevoglucosenone obtained in step a), according to the Baeyer-Villiger reaction, according to one of the following reaction schemes, as a function of the starting material:

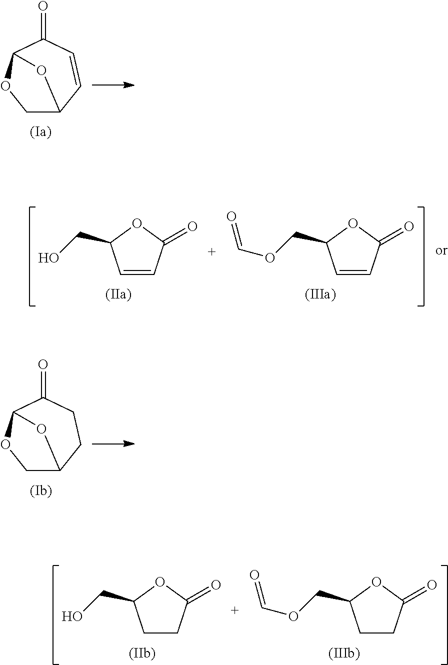

The reaction medium obtained at the end of step b) comprises a mixture of the target compound (IIa) or (IIb) with the corresponding formate (IIIa) or (IIIb).
c) Hydrolysis, in particular acid hydrolysis, of the reaction mixture obtained in step b), in order to transform the formate (IIIa) or (IIIb) contained in this mixture into the corresponding target compound (IIa) or (IIb).

This step may be carried out by any conventional method in itself, for example, by using hydrochloric acid, in particular, in solution in methanol, acetic acid, sulphuric acid, a resin such as AMBERLYST® or even any acid zeolite. The hydrolysis step may alternatively be carried out under basic conditions, according to any conventional method in itself by the person skilled in the art.
d) If applicable, to obtain a compound with general formula (II) where R represents —CH₂—CH₂—, that is to obtain the 4-hydroxymethyl butyrolactone in formula (IIb), if step a) has not been carried out, hydrogenation of the compound (IIa) obtained in step c).

This hydrogenation reaction may be carried out according to any method known to the person skilled in the art, in particular, by catalytic hydrogenation, for example, according to the methods indicated above.

According to the disclosure, step b) for the oxidation of levoglucosenone, or dihydrolevoglucosenone, is carried out by putting a solution of levoglucosenone or dihydrolevoglucosenone in a solvent, in contact with a lipase in the presence of an oxidizing agent and an acyl donor compound.

The oxidation reaction is thereby advantageously catalyzed by enzyme. The benefits are numerous, both from an economic and ecological point of view. In particular, the lipase presents little or no toxicity, in particular, when compared with the metal catalysts and peracids used by the prior art.

In addition, it was discovered that, in a surprising manner, the use of a lipase to catalyze the oxidation step of the levolucosenone or dihydrolevoglucosenone provides high levels of conversion of these substrates in a reduced period of time, in particular, in a much more efficient manner than when the same step is used for cyclohexanone, where the high ring strain is nevertheless similar. Conversion rates of levoglucosenone or dihydrolevoglucosenone exceeding 70% may thereby be obtained in 4 hours or less, and even, in certain conditions, in about two hours, rates of conversion of about 90% were obtained in about 3 hours. From the reaction kinetics point of view, the method according to the disclosure is also more advantageous than the methods proposed by the prior art, while maintaining the advantage of a significant environmental gain.

In addition, it was noted again in an unexpected manner, that at the end of step b) of oxidation, the lipase has a high residual activity, allowing it to be used again for the catalysis of at least one, and even several, subsequent oxidation reactions according to the disclosure. This result is even more unexpected since the oxidation reaction of levoglucosenone or dihydrolevoglucosenone produces, as co-product of the reaction, formic acid, known to have a negative impact on the activity of lipases.

Therefore, to take advantage of this significant residual activity of the lipase at the end of step b) of oxidation of levoglucosenone or dihydrolevoglucosenone, the disclosure advantageously provides, in preferred modes of use, that the method comprises a step of isolating the lipase from the reaction medium after this step b) of oxidation, prior to the implementation of step c) of hydrolysis, in particular, acid hydrolysis.

In an additional advantage of the method according to the disclosure, when step b) of oxidation is carried out from dihydrolevoglucosenone of formula (Ib) above, is the remarkable and surprising regio-selectivity of the reaction leading, with high yields similar to those obtained with levoglucosenone, to the formation of 4-hydroxymethyl butyrolactone of formula (IIb) and the corresponding formate (IIIb).

According to specific means of implementation, the disclosure also complies with the following characteristics, implemented separately or in each of their technically feasible combinations.

In particular, these characteristics aim at reaching the goals set by the disclosure in reducing the cost associated with the use of the method according to the disclosure, as well as limiting the environmental impact.

In particular, the choice of the different preferential characteristics of the disclosure, in particular, the values of the different operational parameters of step b) of the oxidation of levoglucosenone or dihydrolevoglucosenone, indicated below, is carried out according to the disclosure so as to obtain the highest possible rates of conversion of levoglucosenone or dihydrolevoglucosenone, with a reduced reaction time, while maintaining a high residual activity of the enzyme at the end of the reaction, so as to be able to use it again for further reactions, and thereby reduce the cost involved in the use of the method.

The lipase may be of any type. Preferably, lipase B of Candida antartica, known as CaL-B, is used. Other lipases, such as Candida rugosa or Rhizomucor miehei, for example, may also be used.

The lipase may be used in free form in the reaction medium, that is, in a form in which it is not immobilized on a solid support. In particular, the use of lipases in free form provides good yields for the oxidation reaction, in short times, in particular, several hours, while having the advantage that the cost of preparation is low.

In variants of the disclosure, the lipase is used in immobilized form on a solid support so that it may easily be isolated from the reaction medium at the end of step b) of oxidation. For example, a CaL-B lipase immobilized on a polymethyl methacrylate support may be used, such as the lipase commercialized by the company NOVOZYMES® as NOVOZYM® 435.

In especially advantageous means of implementation of the disclosure, the duration of step b) of oxidation of levoglucosenone or dihydrolevoglucosenone is between two and four hours, and preferably between two and three hours. As mentioned above, such a short duration for the reaction provides high rates of conversion of levoglucosenone and dihydrolevoglucosenone.

The solvent used in step b) of oxidation, for the solution of levoglucosenone or dihydrolevoglucosenone, may be of any type compatible with the action of the lipase. It may, for example, consist of an organic solvent, an organic solvent/water mixture, an ionic liquid, a eutectic solvent, a mixture of such solvents, or even supercritical CO₂.

This solvent, for example, may be chosen from toluene, dichloromethane, hexane, etc., or one of their mixtures.

The acyl donor compound may be of any conventional type in itself. In particular, it may consist of a long chain acid, such as a saturated fatty acid with a linear chain in C2 to C30, for example, caprylic acid or capric acid.

In preferred means for the implementation of the disclosure, in step b) of oxidation of levoglucosenone or dihydrolevoglucosenone, the acyl donor compound and the solvent consist of the same product. Preferably, the solvent used for the solution of the levoglucosenone or dihydrolevoglucosenone substrate is thereby an organic solvent, preferably ethyl acetate that, at the same time, ensures the acyl donor function. Other solvents, such as butyl acetate or ethyl propionate, may otherwise be used, alone or a mixture of them or with ethyl acetate.

In particular, ethyl acetate has the advantage that it is biobased and is not toxic for living organisms and the environment.

The oxidizing agent used in step b) of the oxidation of levoglucosenone or dihydrolevoglucosenone is preferentially chosen from among hydrogen peroxide and carbamide peroxide, otherwise known as hydrogen peroxide-urea in a water solution. Such oxidizing agents, in particular, have good stability, so that the risks associated with their industrial use are reduced. Preferentially, the oxidizing agent is oxygen peroxide alone, that is, not associated with urea, which provides especially high rates of conversion.

In step b) of the oxidation of levoglucosenone or dihydrolevoglucosenone, the concentration in oxidizing agents is at least equal to 1 molar equivalent with respect to levoglucosenone or dihydrolevoglucosenone. This concentration is preferably between 1 and 2 molar equivalents with respect to levoglucosenone or dihydrolevoglucosenone. Such a concentration range advantageously provides a high reaction yield, without denaturing the lipase that thereby, can be used again for further reactions. Preferentially, the concentration of oxidizing agents is about 1.2 molar equivalents with respect to levoglucosenone or dihydrolevoglucosenone.

The temperature of step b) of the oxidation of levoglucosenone or dihydrolevoglucosenone preferably ranges from 30° C. to 60° C. in order to ensure the optimum performance of the lipase. Preferably, this temperature is about 40° C. Such a temperature, in particular, provides a rapid kinetics of the oxidation reaction, while limiting the energy expenditure, and thereby the cost, associated with the use of the method according to the disclosure.

For step b) of the oxidation of levoglucosenone or dihydrolevoglucosenone, the levoglucosenone or dihydrolevoglucosenone concentration in organic solvent is, in addition, preferably between 0.5 and 1 mol/L. Concentrations in such a range of values advantageously provides optimum yields for the oxidation reaction.

In specific means for the implementation of the disclosure, the acetic acid and formic acid co-products of the oxidation reaction are eliminated as they are formed, by in situ neutralization or extraction from the reaction medium.

In particularly advantageous means of use of the disclosure, step b) of the oxidation of levoglucosenone or dihydrolevoglucosenone is carried out, in the reaction medium, in the presence of at least one buffer substance, preferably with a pKa between 5 and 9.6, or a mixture of such buffer substances. Such a buffer substance advantageously neutralizes the acetic acid and formic acid co-products of the oxidation reaction, thereby preventing the harmful effect these acids may have on the lipase. In addition, it maintains an optimum pH in the reaction medium for the operation of the latter.

The buffer substance may be a liquid buffer substance, such as a phosphate buffer, conventional in itself

Otherwise, the buffer substance may be a solid buffer substance, preferably with a pKa between 5 and 9.6, and preferentially between 7.2 and 9.6. “Solid buffer substance” refers to a mixture of the two acidic and basic forms of the buffer.

Such a solid form of the buffer substance has several advantages. It enables an easy separation of the buffer substance and the reaction medium. In addition, and in a fully unexpected manner, the use of a solid buffer substance during the oxidation step significantly increases the speed of the oxidation reaction, with respect to conditions without a buffer, and even, to a lesser extent, liquid buffer substances. Therefore, solid buffer substances provide, in two hours of reaction, conversion rates close to 90%.

Any type of solid buffer substance, with an adequate pKa, may be used in the disclosure. Preferably, the solid buffer substance is chosen for its behavior in the reaction medium, in particular, so that it does not form a gel in this medium, so that, on the one hand, it does not disturb the catalytic action of the lipase and, on the other hand, it can be easily separated from the reaction medium at the end of the oxidation reaction. By way of example, it is possible to use a TAPS buffer substance (sulfonic N-[tris(hydroymethyl)methyl]-3-aminopropane acid), or a CAPSO buffer substance (cyclohexylamino-3-hydroxy-2-propanesulfonic-1 acid). Such examples are in no case limiting for the disclosure.

The concentration of solid buffer substance in the reaction medium is preferably between 20 and 100 mg/mL, that is, between 10 and 50 mg/mL of each of the acidic and basic forms of the buffer.

In specific modes for the implementation of the disclosure, for step b) of the oxidation of levoglucosenone or dihydrolevoglucosenone, the quantity of lipase used is between 56 and 1,134 units of lipase per millimole of levoglucosenone or dihydrolevoglucosenone. In a surprising manner, such a small quantity of enzyme provides high rates of conversion of levoglucosenone or dihydrolevoglucosenone in reduced reaction times.

More specifically, when the reaction medium does not have a solid buffer substance, the quantity of lipase used is preferably between 227 and 1,134 units of lipase per millimole of levoglucosenone or dihydrolevoglucosenone. When the reaction medium contains a solid buffer substance, the quantity of lipase used is preferably between 56 and 227 units of lipase per millimole of levoglucosenone or dihydrolevoglucosenone. Such concentration ranges, at a temperature of 40° C., advantageously provide rates of conversion of levoglucosenone or dihydrolevoglucosenone exceeding 70%, in about two hours or about four hours, in the presence or absence of solid buffer substance, respectively.

Steps b) of oxidation and c) of hydrolysis, in particular, acid hydrolysis, of the method in the disclosure may be carried out in a single reactor, without the isolation of the intermediate products. These successive stages may otherwise be separated by an intermediate step of separation of the lipase and, if applicable, of the solid buffer substance, from the reaction medium, optionally followed by a concentration step of this reaction medium prior to the implementation of step c) of hydrolysis, in particular, of acid hydrolysis, of the mixture comprising the compound in formula (II) and the corresponding formate, to obtain the 4-hydroxymethyl butenolide of formula (IIa) or the 4-hydroxymethyl butyrolactone of formula (IIb).

### BRIEF DESCRIPTION OF THE DRAWINGS

The characteristics and advantages of the method in the disclosure appear more clearly in the light of the examples used below, provided by way of illustration and in no way limiting the disclosure, with the support of FIGS. 1A to 7, where:

FIGS. 1A, 1B, and 1C represent the HPLC chromatograms obtained for the samples taken from the reaction medium during the implementation of a step in compliance with the oxidation of levoglucosenone (LGO) into 4-hydroxymethyl butenolide (HBO) and the corresponding formate (FHBO), the samples taken from the reaction medium after 0 hour, 1 hour and 2 hours of reaction, respectively;

FIG. 2 shows a graph illustrating the evolution over time of the rate of conversion of levoglucosenone (LGO) during the implementation of a step of oxidation according to the disclosure, in the following conditions: absence of solid buffer substance at 40° C., absence of solid buffer substance at 60° C., CAPSO solid buffer at 40° C., MOPS solid buffer at 40° C., and TAPS solid buffer at 40° C.;

FIG. 3 shows a graph illustrating the rate of conversion of levoglucosenone (LGO) during the implementation of successive cycles of oxidation reactions according to the disclosure by means of the same lipase, with quantities of lipase in the initial reaction medium of 10% or 20% w/w (113 or 227 units/mmol) with respect to LGO in the presence of a solid buffer;

FIG. 4 presents a graph illustrating the evolution of the rate of conversion of levoglucosenone (LGO) over time on the one hand and that of cyclohexane (CH) on the other hand, during the implementation of a step of oxidation according to the disclosure;

FIG. 5 presents a graph illustrating the evolution of the rate of conversion of levoglucosenone (LGO) over time during the implementation of a step of oxidation according to the disclosure, with hydrogen peroxide (H₂O₂) or hydrogen peroxide-urea (UHP) as oxidizing agent;

FIG. 6 presents a graph illustrating the evolution of the rate of conversion of levoglucosenone (LGO) over time during the implementation of a step of oxidation according to the disclosure, with a type B lipase of Candida Antartica immobilized on solid support (S) or a type B lipase of Candida Antartica in free form, with (LTpn) or without (L) liquid buffer in the reaction medium; and

FIG. 7 presents a graph illustrating the evolution of the rate of conversion of levoglucosenone (LGO) over time during the implementation of a step of oxidation according to the disclosure, with a type B lipase of Candida Antartica immobilized on solid support (S), in the presence of a solid CAPSO, MOPS or TAPS buffer, or a type B lipase of Candida Antartica in free form, with the presence of a liquid buffer in the reaction medium (LTpn).

### DETAILED DESCRIPTION

### EXAMPLE 1

### Synthesis of 4-hydroxymethyl butenolide

4-hydroxymethyl butenolide of formula (IIa) is prepared from levoglucosenone (LGO) according to a specific means of implementation of the method according to the following disclosure, called “one-pot.”

Oxidation

In a reactor, an aqueous solution of 30% hydrogen peroxide H₂O₂ (2.57 mmol, 0.26 mL, 1.2 eq. with respect to LGO) is added in one portion to a suspension of LGO (270 mg, 2.14 mmol) and CaL-B lipase (NOVOZYM® 435, 75 mg, 315 U/mmol LGO) in ethyl acetate (3 mL) with stirring at room temperature in a planar stirring incubator. In this example, as for all of the following examples, 1 g of NOVOZYM® 435 corresponds to 9000 units of CaL-B lipase (activity measured after immersion of the enzyme in ethyl acetate). The reaction mixture is stirred at 40° C. for 4 hours and then evaporated to dryness.

Acid Hydrolysis

Concentrated hydrochloric acid (5 mmol, 0.4 mL) is added to a solution of this crude mixture at room temperature. The reaction mixture is heated while stirring for 8 to 16 hours, so as to convert the formate (IIIa) into the corresponding alcohol (IIa). The reaction mixture is evaporated to dryness with silica gel. The crude product is purified by chromatography on silica gel (elution with 75% to 100% ethyl acetate in cyclohexane) to obtain pure 4-hydroxymethyl butenolide (IIa) (175 mg, 72%).

¹H NMR (CDCl₃): δ 7.53 (dd, J=1.5 and 5.7 Hz, 1 H), 6.2 (dd, J=1.5 and 5.7 Hz, 1 H), 5.17 (m, 1 H), 4.0 (d, J=3.6 and 12.0 Hz, 1 H), 3.80 (dd, J=3.6 and 12.0 Hz, 1 H)

¹³C NMR (CDCl₃): δ 173.5(s), 154.0 (d), 122.8 (d), 84.3 (d), 62.2 (t)

As a variant, at the end of the step of LGO processing with the lipase, the latter is separated from the reaction medium prior to the step of evaporation to dryness of the medium. The acid hydrolysis is then carried out as described above. Pure 4-hydroxymethyl butenolide is also obtained with a similar yield of 72%.

In other variants of the method according to the disclosure, the step of acid hydrolysis is directly carried out on the reaction medium obtained after the oxidation step, without first carrying out the evaporation to dryness step. Whether the lipase is eliminated from the reaction medium by filtration or not, in such variants of the disclosure, the yield of this reaction is similar to that obtained for the means of implementation described above in detail, that is, about 72%.

### EXAMPLE 2

### Synthesis of 4-hydroxymethyl butyrolactone

4-hydroxymethyl butyrolactone, of formula (IIb) is prepared from levoglucosenone (LGO) according to any of the variants of the method according to the following disclosure.

2.1/Route 1

Catalytic Hydrogenation

Pd/C (10% w/w, 500 mg) is added to a solution of (-)-levoglucosenone LGO (5 g, 39.7 mmol) in ethyl acetate (50 mL) at room temperature. The suspension while stirring is degassed three times under vacuum/nitrogen. The suspension is then hydrogenated by an atmosphere of hydrogen at room temperature until the initial product is fully consumed, that is, about 4 hours. The crude mixture is filtered through a celite buffer and the filtrate is concentrated to dryness with silica gel. The crude product is purified by chromatography on silica gel (elution with 10 to 60% ethyl acetate in cyclohexane), to obtain the pure dihydrolevoglucosenone of formula (Ib) (2H-LGO) (colorless oil, 4.4 g, 87%).

¹H NMR (CDCl₃): δ 5.10 (s, 1 H), 4.7 (m, 1 H), 4.09 (dd, J=0.8 and 7.5 Hz, 1 H), 3.98 (ddd, 5.0 and 7.0 Hz, 1 H), 2.66 (m, 1 H), 2.45-2.22 (m, 2 H), 2.10-1.97 (m, 1 H)

¹³C NMR (CDCl₃): δ 200.2(s), 101.5 (d), 73.1 (d), 67.5 (t), 31.1 (t), 29.9 (t)

Oxidation

In a reactor, an aqueous solution of 30% hydrogen peroxide H₂O₂ (9.3 mmol, 0.97 mL, 1.2 eq. with respect to 2H-LGO) is added in one portion to a suspension of 2H-LGO (1 g, 7.8 mmol) while stirring, in addition containing the lipase (NOVOZYM® 435, 100 mg, 340 U/mmol 2H-LGO) in ethyl acetate (10 mL) at room temperature. The reaction mixture is stirred at 40° C. for 4 hours and then evaporated to dryness.

Acid Hydrolysis

Concentrated hydrochloric acid (12 mmol, 1 mL) is added to a solution of this crude mixture in methanol (10 mL) at room temperature. The reaction mixture is heated while stirring for 8 to 16 hours, so as to convert the formate (IIIb) into the corresponding alcohol (IIb). The reaction mixture is evaporated to dryness with silica gel. The crude product is purified by chromatography on silica gel (elution with 75% to 100% ethyl acetate in cyclohexane) to obtain pure 4-hydroxymethyl butyrolactone (IIb) (750 mg, 83%).

¹H NMR (CDCl₃): δ 4.64 (m, 1 H), 3.92 (dd, J=2.7 and 12.6 Hz, 1 H), 3.66 (dd, J=4.5 and 12.6 Hz, 1 H), 2.72-2.49 (m, 3 H (2 H +OH)), 2.35-2.09 (m, 2 H)

¹³C NMR (CDCl₃): δ 177.7(s), 80.8 (d), 64.1 (t), 28.7 (t), 23.1 (t)

2.2/Route 2

The 4-hydroxymethyl butenolide (IIa) obtained in Example 1 undergoes catalytic hydrogenation as follows:

Pd/C (10% w/w, 250 mg) is added to a solution of 4-hydroxymethyl butenolide (1.4 g, 12.3 mmol) in ethyl acetate (15 mL) at room temperature. The suspension, while stirring, is degassed three times under a vacuum/nitrogen. The suspension is then hydrogenated by a hydrogen atmosphere at room temperature for 4 hours. The crude mixture is filtered through a celite buffer and the filtrate is concentrated to dryness with silica gel. The crude product is purified by chromatography on silica gel (elution with a gradient ranging from 75 to 100% ethyl acetate in cyclohexane), to obtain the pure 4-hydroxymethyl butyrolactone in formula (IIb) (1.19 g, 82%).

¹H NMR (CDCl₃): δ 4.64 (m, 1 H), 3.92 (dd, J=2.7 and 12.6 Hz, 1 H), 3.66 (dd, J=4.5 and 12.6 Hz, 1 H), 2.72-2.49 (m, 3 H), 2.35-2.09 (m, 2 H)

¹³C NMR (CDCl₃): δ 177.7(s), 80.8 (d), 64.1 (t), 28.7 (t), 23.1 (t)

Both means of synthesis 1 and 2 above provide, in a regioselective manner and with high yields, the 4-hydroxymethyl butyrolactone (IIb), the structure of which is confirmed by proton and carbon nuclear magnetic resonance (NMR).

### EXAMPLE 3

### Kinetic Monitoring of the Oxidation Reaction by HPLC

HPLC Protocol

A 10 μL sample of the reaction medium is diluted in 1.5 mL of acetonitrile. The mixture is passed through a 0.2 μm PTFE filter and then injected in the HPLC chromatograph.

The analyses are carried out on a THERMO SCIENTIFIC® SYNCRONIS™ aQ column (250*4.6 mm, 5 μm) in the following conditions: injection volume 10 μL; oven temperature 30° C.; elution method: isocratic 85/15 water/acetonitrile from 0 to 5 minutes, from 5 to 10 minutes gradient of 85/15 to 90/10 water/acetonitrile, from 10 to 15 minutes isocratic 90/10 water/acetonitrile, from 15 to 20 minutes gradient of 90/10 to 85/15 water/acetonitrile; recording of the spectrum at 220 nm.

Kinetic Monitoring

The kinetics of the oxidation reaction, carried out according to the disclosure, of levoglucosenone (LGO) into 4-hydroxymethyl butenolide (IIa) (HBO) is followed by HPLC.

499 mg of LGO (3.96 mmol) is dissolved in 5.3 mL of ethyl acetate (LGO concentration: 0.75 mol/l). TAPS solid buffer (150 mg) and TAPS sodium salt (149 mg) are added to this solution, then 77 mg of Cal-B lipase (NOVOZYM® 435, 15% weight, 170 U/mmol LGO) and finally 0.33 mL of 50% hydrogen peroxide in water (1.2 eq/LGO). The medium is stirred at 40° C.

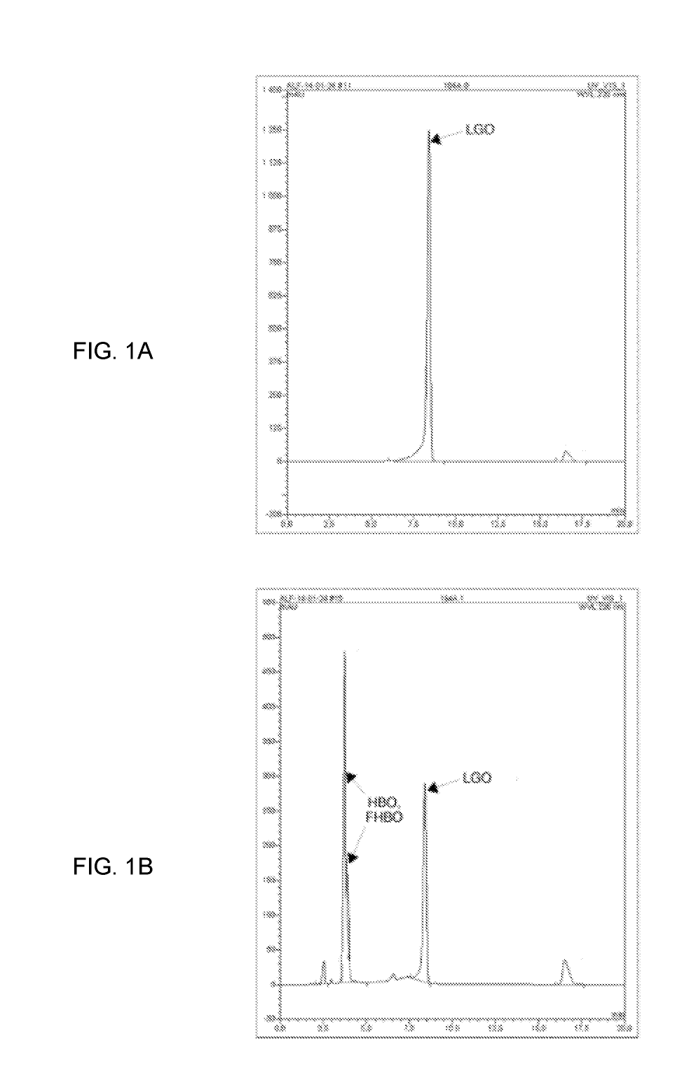

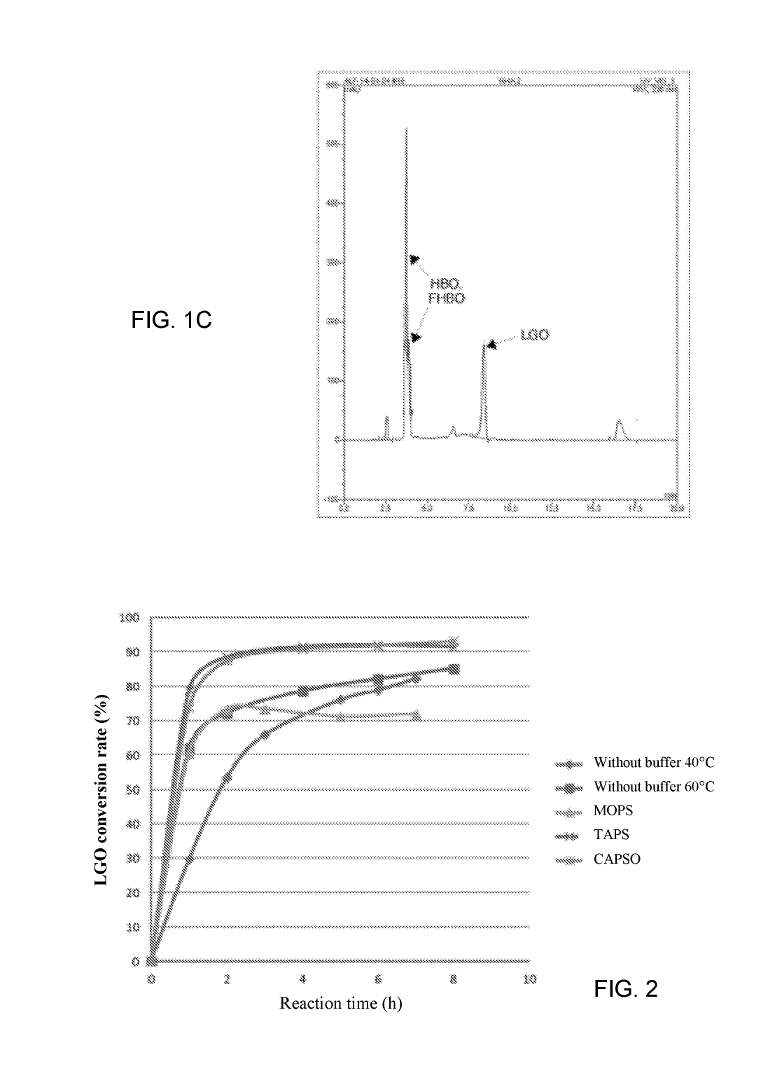

10 μL samples are taken from the reaction medium at different intervals and analyzed by HPLC according to the protocol described above. The HPLC chromatograms obtained at reaction times T=0 h, T=1 h and T=2 h are presented in FIGS. 1A, 1B, and 1C, respectively.

The peaks corresponding to the different compounds are attributed by comparison with the HPLC chromatograms obtained from pure products, commercially available or by other means and purified in-house, according to the retention times:
LGO: 8.40 minutes
HBO and corresponding FHBO formate: 3.73 and 3.87 minutes

To determine the rate of conversion of LGO at each time, the area under the peak attributed to LGO is measured and indicated on a standard curve made by HPLC analysis of LGO solutions of known concentration. The rate of conversion of LGO is deducted from the value in mass thereby determined, by comparison with the initial mass of LGO.

The results obtained are provided in Table 1 below.

TABLE 1
Rate of conversion of LGO after different reaction times, by HPLC
analysis.

| | Reaction time (h) | | |
|---|---|---|---|
| | 0 | 1 | 2 |
| Area under the HPLC peak of LGO | 0.7422 | 0.1528 | 0.0845 |
| Rate of conversion of LGO (%) | 0 | 79 | 88 |

### EXAMPLE 4

### Study of the Presence of a Buffer Substance on the Rate of Conversion

Oxidation reactions of levoglucosenone (LGO), to obtain a mixture of 4-hydroymethyl butenolide (IIa) (HBO) and the corresponding formate (IIIa), are carried out according to the disclosure under the following conditions.

In a 0.75 mol/L solution of LGO in ethyl acetate, CaL-B lipase (NOVOZYM® 435, 50 mg/mmol of LGO, 450 U/mmol LGO) is added, followed by 1.2 eq. of 50% hydrogen peroxide in water. The reaction medium is stirred at 40° C. or 60° C. for 24 hours.

Reactions are also carried out with the following solid buffers, added to the reaction medium at the beginning of the reaction, with a concentration of 20 mg/mL for each of the acidic and basic forms: commercially available MOPS (pKa 7.2), TAPS (pKa 8.4) or CAPSO (pKa 9.6).

For each of the solid buffers, as well as for a reaction medium without buffer, at 40° C. and at 60° C., the conversion rate of LGO is monitored over time and assessed by HPLC, according to the procedure in Example 3, from the measurement of the area under the HPLC peak attributed to LGO (retention time 8.40 minutes).

The results obtained are provided in FIG. 2. LGO conversion rates exceeding 70% are obtained, for the reaction without buffer, in less than 4 hours at 40° C. and in less than 2 hours at 60° C., and, for all the solid buffers tested, in less than 2 hours at 40° C. The solid CAPSO and TAPS buffers even provide, in 2 hours of reaction, conversion rates that are approximately equal to 90%. The slightly inferior performance of the MOPS buffer may be attributed to the fact that this substance forms a gel in the reaction medium that may hamper the action of the lipase.

To compare the effect on the LGO oxidation reaction of a solid buffer according to the disclosure, a procedure in the same reaction conditions is carried out by way of comparative example but with the addition of a liquid buffer in the reaction medium at the beginning of the reaction, more precisely, a phosphate buffer of pKa 7.2 at the rate of 1 mL of buffer for 4 mL of ethyl acetate. After 24 hours of reaction, a lipase conversion rate of 43% is obtained.

This result clearly demonstrates the high and surprising effectiveness of solid buffers in the implementation of the method according to the disclosure.

### EXAMPLE 5

### Measurement of the Residual Activity of the Lipase at the End of the Oxidation Reaction

5.1/Measurement by Transformation of Lauric Acid

LGO oxidation reactions are carried out according to the disclosure with the following operational parameters. The initial LGO concentration of ethyl acetate equals 0.75 mol/L. The reaction is carried out at 40° C. The quantity of CaL-B lipase is 226.8 units per mmol of LGO. The oxidizing agents used are hydrogen peroxide (H₂O₂) or hydrogen peroxide-Urea (H₂O₂-urea) with a concentration of 1.2 molar equivalents with respect to LGO. Different reaction times are tested: 4 hours, 6 hours, 8 hours, 16 hours, and 24 hours.

At the end of the reaction, the lipase is isolated from the reaction medium by filtration, washed with ethyl acetate, water and then hexane, dried for 1 hour at 40° C. and then one night at room temperature in a desiccator under reduced pressure, and finally is put through the following test.

The residual activity of the lipase is measured by gas phase chromatography coupled with mass spectrometry (GC-MS), by determining the conversion rate of lauric acid into propyl laurate.

For this purpose, a solution of substrate with the following composition is prepared: 74.65% (w/w) lauric acid, 22.37% (w/w) 1-propanol, 2.98% (w/w) water. This mixture is liquefied at 60° C. 14 mg of lipase are transferred to a flask, placed at 60° C. 5 g (6 mL) of the substrate solution are added to the flask.

After 15 minutes of reaction, four samples of 2 μL each are taken and placed in flasks for gas phase chromatography (GC) of known weight. 1 mL of hexane is added to each flask and then the quantity of lauric acid converted by the lipase is assessed by GC-MS as follows:
GC: flask containing at least 500 μL of liquid; injection of 1 μL, in split mode 40:1; injector temperature 280° C.; gas vector: H₂, 1.2 mL/minute at constant flow; oven temperature: 60° C. for 1 minute and then temperature gradient of 20° C./minute until 325° C. and maintenance at 325° C. for 5 minutes; temperature of the transfer line: 280° C.
MS: solvent delay 1 minute; temperature of the source: 230° C.; quad temperature: 150° C.; scan range 30 to 350 amu.

A standard mass range is determined for each sample passage (from 0.1 g/L to 2.8 g/L or 0.2 mg/g to 4.5 mg/g). The mass of the lauric acid remaining in each sample is determined by comparison with the standard range.

The activity of the enzyme, in PLU/g, is determined by the equation:

Activity=[(Mᵢ−M_{f})/(W×t)]×1000
Where Mᵢ represents the initial number of mmoles of lauric acid
M_{f} represents the final number of mmoles of lauric acid
W represents the quantity of lipase (g)
t represents the reaction time (minutes)
One PLU unit is defined as the quantity of enzyme that, in standard conditions of 60° C. and 15 minutes of reaction, forms 1 μmol of propyl laurate per minute.
M_{f} is calculated from the corresponding areas of the peak in GC-MS and by means of a calibration curve.

When there is a loss of activity of the lipase when coming into contact with the ethyl acetate (AcOEt) and where this loss of activity is not prolonged over time, a residual activity of 100% is defined for a suspension of the lipase in ethyl acetate.

The results obtained, with the different conditions of the LGO oxidation reaction according to the disclosure, are presented in Table 2 below.

TABLE 2
Residual activity of the lipase after implementation of an LGO
oxidation reaction according to the disclosure.

| | Activity | Residual activity |
|---|---|---|
| Conditions | (PLU/g) | (%) |
| Initial CaL-B (tailpipe) | 16763 | |
| Initial CaL-B in AcOEt | 9593 | 100.0 |
| (H₂O₂), 4 hours reaction | 8116 | 84.6 |
| (H₂O₂), 6 hours reaction | 7532 | 78.6 |
| (H₂O₂), 8 hours reaction | 7132 | 74.4 |
| (H₂O₂), 16 hours reaction | 3594 | 37.5 |
| (H₂O₂-urea), 6 hours reaction | 7947 | 82.9 |
| (H₂O₂-urea), 24 hours reaction | 5069 | 52.9 |

The residual activity of the lipase remains high even after long reaction times. After 6 hours of reaction with LGO, this residual activity exceeds 78%, no matter what oxidizing agent is used.

5.2/Re-Use of the Lipase in a Method According to the Disclosure

A first LGO oxidation reaction according to the disclosure is carried out in the following conditions. The concentration of LGO in ethyl acetate equals 0.67 mol/L. The following are added to this solution: 113 or 227 units of Cal-B lipase per mmol of LGO (10% or 20% in weight of lipase with respect to the weight of LGO, respectively), 1.2 equivalents of hydrogen peroxide (50% in water), and 20 mg/mL of each of the acidic and basic forms of HEPES solid buffer (pKa 7.5). The reaction is carried out while stirring at 40° C. for 2 hours.

At the end of the reaction, 10 μL of reaction medium are taken and analyzed by HPLC according to the protocol described in Example 2 above.

The lipase and solid buffer are then separated from the reaction medium by filtration and washed with ethyl acetate.

They are then used again for a second reaction cycle, in the same conditions as those described above, then in a third and finally a fourth reaction cycle.

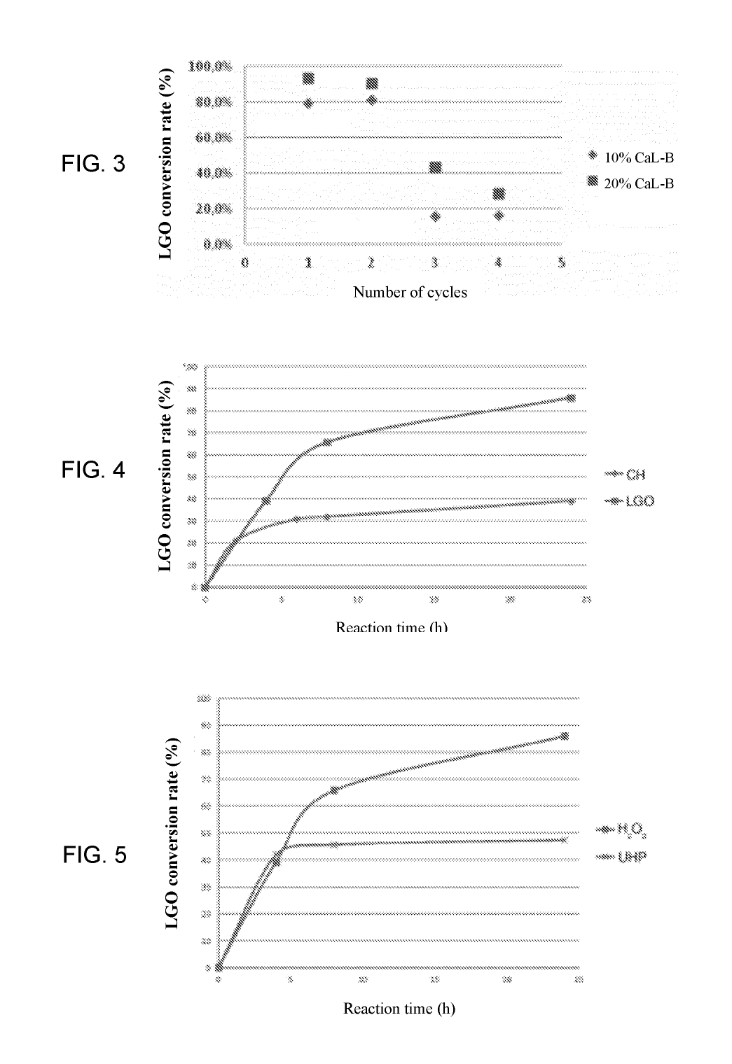

At the end of each cycle, the LGO conversion rate is determined by HPLC analysis. The results obtained are presented in FIG. 3. This figure shows that it is possible to carry out, with the same lipase, a second LGO oxidation reaction cycle without any loss of yield. A third cycle and even a fourth cycle are also possible although the yields are lower.

### EXAMPLE 6

### Variation of the Operating Conditions for the Oxidation of Levoglucosenone

Step b) of the method according to the disclosure, for the oxidation of levoglucosenone (LGO), is implemented as described in Example 1, by varying the different operating parameters as indicated in Table 3 below. After 2 hours of reaction at 40° C., the following are found: the LGO conversion rate, as indicated in Example 4 above, and the residual activity of the lipase, as indicated in Example 5 above.

The results obtained are presented in Table 3 below.

TABLE 3
LGO conversion rate and residual activity of the lipase after LGO
oxidation reactions according to the different means to implement
the method in the disclosure

| | Experiment | | | | |
|---|---|---|---|---|---|
| | 1 | 2 | 3 | 4 | 5 |
| H₂O₂ | 1.4 | 1.5 | 1.4 | 1.4 | 1.5 |
| (eq./LGO) | | | | | |
| Quantity of | 675 | 1143 | 1089 | 657 | 1080 |
| Cal-B (U) | | | | | |
| Quantity of | 166 | 290 | 271 | 166 | 273 |
| CaL-B | | | | | |
| (U/mmol LGO) | | | | | |
| Volume of | 5.3 | 4 | 5.3 | 5.3 | 8 |
| ethyl acetate | | | | | |
| (mL) | | | | | |
| Initial LGO | 0.768 | 0.983 | 0.757 | 0.748 | 0.494 |
| concentration | | | | | |
| (mol/L) | | | | | |
| Solid buffer | TAPS | MOPS | TAPS | CAPSO | MOPS |
| Weight of solid | 112.6 | 86.6 | 112.6 | 112.6 | 166.6 |
| buffer (each of | | | | | |
| the acidic | | | | | |
| and sodium | | | | | |
| forms) (mg) | | | | | |
| Final LGO | 0.048 | 0.083 | 0.066 | 0.075 | 0.092 |
| concentration | | | | | |
| (mol/L) | | | | | |
| LGO conversion | 93.8 | 91.6 | 91.3 | 90.0 | 81.3 |
| rate (%) | | | | | |
| Residual lipase | 74.9 | 44.8 | 50.6 | 68.9 | 85.3 |
| activity (%) | | | | | |

These results clearly show that, after 2 hours of reaction, all the means of implementation of the method according to the disclosure provide levoglucosenone conversion rates exceeding 80%, and even up to 94%. At the end of the reaction, the lipase also has a residual activity of at least 44% and up to 85% according to the conditions, so much so that it can effectively be re-used in additional oxidation reactions.

### EXAMPLE 7

### Comparative example—Oxidation of Levoglucosenone and Cyclohexanone

The oxidation of levoglucosenone (LGO) or cyclohexanone is carried out as described in Example 1 above, with the following operating parameters.

In a 0.75 mol/L solution of substrate (cyclohexanone of LGO) in ethyl acetate, CaL-B lipase (NOVOZYM® 435, 115 U/mmol of substrate) is added, followed by 1.2 eq. of 50% hydrogen peroxide in water. The reaction medium is stirred at 40° C.

The rate of conversion of each substrate is monitored over time and assessed by HPLC, according to the operating protocol in Example 3 above, by measurement of the area under the HPLC peak attributed to the substrate.

The results obtained are presented in FIG. 4. The rate of conversion obtained for levoglucosenone is significantly higher than that obtained for cyclohexanone, as of short reaction times, in spite of the fact that the ring stress of these molecules is similar. This result clearly shows the high and surprising efficacy of the method according to the disclosure for the oxidation of levoglucosenone.

### EXAMPLE 8

### Variation of the Oxidizing Component

Step b in the oxidation of levoglucosenone (LGO) is carried out as described in Example 1 above, with the following operating parameters, both with an oxidizing agent of hydrogen peroxide (H₂O₂) or hydrogen peroxide-Urea (UHP).

In a 0.75 mol/L solution of LGO in ethyl acetate, CaL-B lipase (NOVOZYM® 435, 115 U/mmol of substrate) is added, followed by 1.2 eq. of oxidizing agent (50% hydrogen peroxide in water or UHP). The reaction medium is stirred at 40° C.

In each experiment, the rate of conversion is monitored over time and assessed by HPLC, according to the operating protocol in Example 3 above, by the measurement of the area under the HPLC peak attributed to LGO.

The results obtained are provided in FIG. 5. High rates of conversion are obtained for the two oxidizing agents, although hydrogen peroxide is more efficient than hydrogen peroxide-urea.

### EXAMPLE 9

### Implementation of Lipase in Free Form

9.1/Experiment 1

Step b/of the oxidation of levoglucosenone (LGO) is carried out with CaL-B enzyme immobilized on solid support (NOVOZYM® 435) and the lipase B of Candida antartica in free form CaL-B L, commercialized by LIPOZYME® by NOVOZYMES®.

In a 0.75 mol/L solution of LGO in ethyl acetate, the lipase (115 U/mmol of substrate) is added, followed by the oxidizing agent (50% hydrogen peroxide in water) (1.2 eq. for the lipase immobilized on solid support and 1 eq. for the lipase in free form). The reaction medium is stirred at 40° C.

An experiment is also carried out for the lipase in free form, by including a phosphate buffer of pKa=7 (KH₂PO₄/NaOH) in the reaction medium.

For each experiment, the rate of conversion is monitored over time and assessed by HPLC, according to the operating protocol in Example 3 above, by measurement of the area under the HPLC peak attributed to LGO.

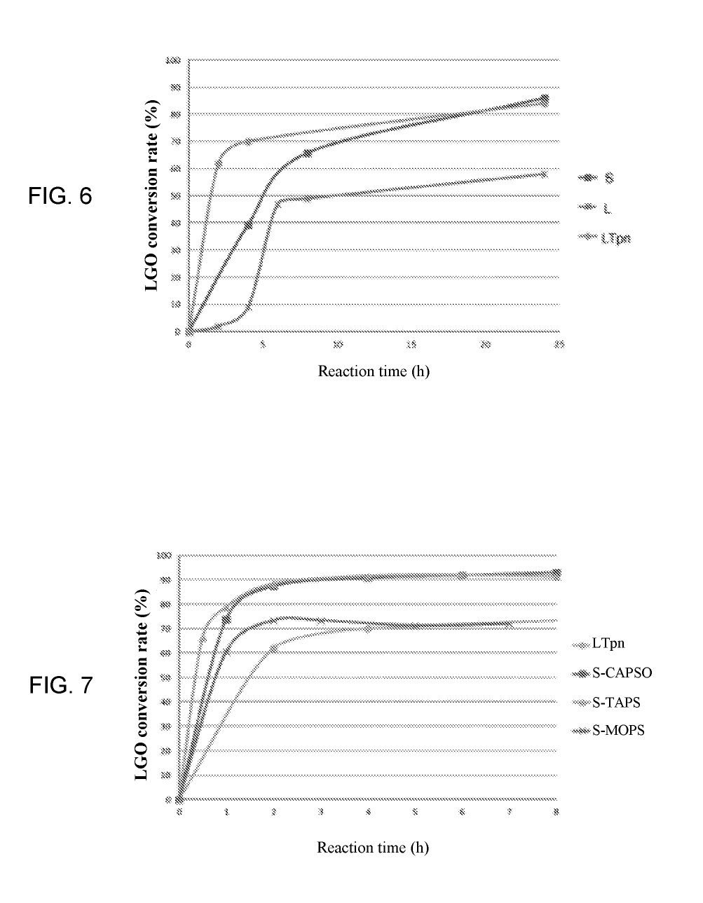

The results obtained are provided in FIG. 6. High rates of conversion are obtained with all of the conditions tested, even though the load in lipase in the reaction medium is relatively low (115 PLU/mmol substrate). When the lipase is used in free form in the presence of liquid buffer, especially high rates of conversion are obtained in short times, similar to those obtained for the lipase immobilized on solid support.

9.2/Experiment 2

Step b/of the oxidation of levoglucosenone (LGO) is carried out with CaL-B enzyme immobilized on solid support (NOVOZYM® 435) and the lipase B of Candida antartica in free-form CaL-B L, commercialized as LIPOZYME® by NOVOZYMES®.

In a 0.75 mol/L solution of LGO in ethyl acetate, the lipase (115 U/mmol of lipase in free form, and 115 U/mmol of LGO for the lipase on solid support) is added, followed by the oxidizing agent (50% hydrogen peroxide in water) (1.2 eq. for the lipase immobilized on solid support, and 1 eq. for the lipase in free form).

A phosphate buffer of pKa=7 (KH₂PO₄/NaOH) is added to the reaction medium for the lipase in free form.

For the lipase on solid support, the different solid buffers are used at a concentration of 20 mg/mL for each of the acidic and basic forms: commercial MOPS (pKa 7.2), TAPS (pKa 8.4) or CAPSO (pKa 9.6).

In all cases, the buffers are added to the reaction medium at the beginning of the reaction.

The reaction medium is stirred at 40° C.

In each experiment, the rate of conversion is monitored over time and assessed by HPLC, according to the operating protocol in Example 3 above, from the measurement of the area under the HPLC peak attributed to LGO.

The results obtained are provided in FIG. 7. High rates of conversion are observed with all of the conditions tested, even though the load in lipase in the reaction medium is relatively low. When the lipase is used in free form in the presence of liquid buffer, a very interesting rate of conversion is obtained (70% conversion) in little time (only 4 hours), with the additional advantage that the cost of the reagents is limited.

### EXAMPLE 10

### Synthesis of 4-hydroxymethyl butenolide from levoglucosenone by means of free-form lipases

4-hydroxymethyl butenolide of formula (IIa) is prepared from levoglucosenone (LGO) according to the specific implementation of the method according to the disclosure.

The following lipases are tested: Lipozyme® Candida antartica type B Cal-B L, Lipase AY “Amano” 30SD-K Candida rugosa, and Lipase MER “Amano.”

The operating protocol is as follows:

In turn, add the following to a 50 mL baffled Erlenmeyer flask or a 250 mL balloon flask:
levoglucosenone (LGO, 504 mg, 4 mmol),
ethyl acetate (5.3 mL),
lipase: either Lipozyme® Candida antartica type B Cal-B L (0.1 to 0.3 mL or 115 U to 350 U, activity 5000 LU/g), or Lipase AY “Amano” 30SD-K Candida rugosa (13 mg or 115 U, activity 30000 U/g), or Lipase MER “Amano” (53 mg or 115 U, activity 7500 LU/g).
50% hydrogen peroxide in water (1 eq., 0.27 mL),
with/without potassium dihydrogen phosphate buffer (KH₂PO₄/NaOH) pH 7 (0.25 mL).

The reaction mixture is stirred magnetically or in an incubator (Thermo MAXQ 150 RPM) at 40° C. for 24 hours.

The reaction medium then undergoes a step of acid hydrolysis as described in Example 1 above.

With each of the enzymes and conditions tests, 4-hydroxymethyl butenolide is obtained with a yield ranging from 60% to 80%.

1. A method for transforming levoglucosenone into a compound with the general formula (II): 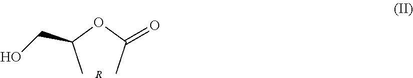
where R represents —CH═CH— or —CH₂—CH₂—, comprising the following steps:
a) if applicable, to obtain a compound of general formula (II) where R represents —CH₂—CH₂—, hydrogenation of levoglucosenone to form dihydrolevoglucosenone,
b) oxidation of the levoglucosenone or dihydrolevoglucosenone obtained in step a),
c) hydrolysis of the reaction mixture obtained in step b),
d) if applicable, to obtain a compound of general formula (II) where R represents —CH₂—CH₂—, if step a) is not carried out, hydrogenation of the compound obtained in step c),
wherein the oxidation of the levoglucosenone or dihydrolevoglucosenone further comprises putting in contact a solution of levoglucosenone or dihydrolevoglucosenone in a solvent, with a lipase in the presence of an oxidizing agent and an acyl donor compound.

2. The method according to claim 1, wherein the quantity of lipase used in the oxidation of the levoglucosenone or dihydrolevoglucosenone is between 56 and 1,134 units of lipase per millimole of levoglucosenone or dihydrolevoglucosenone.

3. The method of claim 1, wherein the lipase is a B lipase of Candida antartica.

4. The method of claim 1, wherein the duration of the oxidation of levoglucosenone or dihydrolevoglucosenone is between two and four hours.

5. The method of claim 1, wherein the acyl donor compound and the solvent consist of the same product.

6. The method of claim 1, wherein the oxidizing agent comprises at least one of hydrogen peroxide and carbamide peroxide, in solution in water.

7. The method of claim 1, wherein a concentration of the oxidizing agent is at least equal to 1 molar equivalent with respect to the levoglucosenone or dihydrolevoglucosenone.

8. The method of claim 1, wherein the oxidation of levoglucosenone or dihydrolevoglucosenone is carried out at a temperature between 30° C. and 60° C.

9. The method of claim 1, wherein a concentration of the levoglucosenone or dihydrolevoglucosenone in the solvent is between 0.5 and 1 mol/L.

10. The method of claim 1, wherein the oxidation of levoglucosenone or dihydrolevoglucosenone is carried out in the presence of at least one solid buffer in the reaction medium.

11. The method of claim 10, wherein a concentration of the solid buffer in the reaction medium is between 20 and 100 mg/mL.

12. The method of claim 1, wherein the lipase is isolated from the reaction medium after the oxidation of levoglucosenone or dihydrolevoglucosenone prior to the implementation of the hydrolysis of the reaction mixture.

13. The method of claim 1, wherein the oxidation of levoglucosenone or dihydrolevoglucosenone is carried out in the presence of at least one liquid buffer in the reaction medium.

14. The method of claim 1, wherein the lipase is immobilized on a solid support.

15. The method of claim 1, wherein the lipase is in free form in the reaction medium.

16. The method of claim 4, wherein the duration of the oxidation of levoglucosenone or dihydrolevoglucosenone is between two and three hours.

17. The method of claim 5, wherein the acyl donor compound and the solvent consist of ethyl acetate.

18. The method of claim 7, wherein a concentration of the oxidizing agent is at between 1 and 2 molar equivalents with respect to the levoglucosenone or dihydrolevoglucosenone.

19. The method of claim 8, wherein the oxidation of levoglucosenone or dihydrolevoglucosenone is carried out at a temperature of 40° C.

20. The method of claim 1, wherein:
the duration of the oxidation of levoglucosenone or dihydrolevoglucosenone is between two and four hours;
the acyl donor compound and the solvent consist of the same product;
the oxidizing agent comprises at least one of hydrogen peroxide and carbamide peroxide;
a concentration of the oxidizing agent is at least equal to 1 molar equivalent with respect to the levoglucosenone or dihydrolevoglucosenone;
the oxidation of levoglucosenone or dihydrolevoglucosenone is carried out at a temperature between 30° C. and 60° C.; and
a concentration of the levoglucosenone or dihydrolevoglucosenone in the solvent is between 0.5 and 1 mol/L.
